(19) United States
(12) Patent Application Publication
(10) Pub. No.: US 2003/0236655 A1
(43) Pub. Date: Dec. 25, 2003

(54) Method and system for computationally estimating transition geometry between reactant and product structures

(76) Inventors: Niranjan Govind, San Diego, CA (US); Robert Dominic King-Smith, San Diego, CA (US); George Fitzgerald, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/180,957

(22) Filed: Jun. 24, 2002

(51) Int. Cl. G06G 7/48; G06G 7/58; G06F 19/00; G01N 33/48; G01N 33/50

(52) U.S. Cl. 703/12

(57) ABSTRACT

A method of estimating transition state energies includes minimizing a sum involving computed atomic coordinates and interatomic distances. For periodic systems, terms of the sum are weighted according to the unit cell in which different atoms reside. For these periodic systems, the sum may be truncated by not including terms corresponding to atom pairs which have a separation distance which is greater than a threshold amount.

### BACKGROUND OF THE INVENTION

1. Field of the Invention

The invention relates to transition states between reactant and product structures, and more specifically, to a method of estimating a transition point energy of a chemical reaction.

2. Description of the Related Art

The “transition state” of a chemical reaction is the atomic configuration having the highest energy through which the system must pass as the molecules transform from reactants to products. For example, FIG. 1 illustrates the transition of two reactants, A₂ and B₂, through an intermediate transition state, A₂B₂, to a product, 2AB. FIG. 1 also includes a potential energy graph illustrating the change in total system energy as the transition from reactants to product occurs. When the molecules A₂ and B₂ collide, the configuration energy of the system increases until a maximum is reached at transition point 102. The transition point 102 marks the top of the potential energy curve and represents the amount of energy necessary in order for the chemical reaction to occur. After the reaction takes place, the potential energy decreases as the product is formed and the two AB molecules relax to a lower energy configuration.

In the field of reaction chemistry, feasibility and reaction rates of chemical reactions depend on this transition state energy and structure. Therefore, researchers in the field often need to accurately calculate transition states. Many scientists at chemical, petrochemical and pharmaceutical companies, and at universities perform active research in this area of computational chemistry.

An article by Halgren and Lipscomb [T. A. Halgren and W. N. Lipscomb, Chem. Phys. Lett. 49, 225 (1977).], which is hereby incorporated by reference for all purposes, introduced the linear synchronous transit (LST) and quadratic synchronous transit (QST) methods for searching for the transition states of molecules. In the LST approach the user specifies reactant and product structures on each side of an energy barrier. The method makes a linear geometric interpolation between the reactant and product structures, and a series of energy evaluations are performed to search for the maximum energy along this path. The maximum energy structure lying on the LST path gives an estimate of the transition state structure and provides an upper bound to the transition state energy.

Halgren and Lipscomb went on to propose a method for refining the LST transition state structure in which an energy minimization in a direction orthogonal to the LST path is used to yield a lower energy intermediate structure. A quadratic interpolation is then performed using the reactant, product and intermediate structure to define the QST path. Single point energy calculations are performed to obtain the maximum energy on the QST path, and the maximum energy structure provides a second (lower) upper bound to the transition state energy along with a refined estimate for the transition state geometry.

FIG. 2 is a schematic illustrating the Halgren/Lipscomb LST and QST approach to transition state searching. As stated above, the LST path 110 is a linear path yielding an upper bound of the transition state energy as the structure changes from reactant state 120 on the energy surface to product state 130 on the energy surface. After the LST path 110 has been determined, a series of single point energy evaluations are performed to search for the maximum energy point 112 along the LST path 110. Thereafter, a single energy minimization in a direction orthogonal to the LST path 110 is performed. More particularly, minimization path 140 advances from the maximum energy point 112 on the LST path 110 “downhill” toward lower energy configurations. However, as can be seen from the contours of FIG. 2, minimization path 140 may deviate from the ridge leading to the saddle point. Therefore, according to one embodiment of the Halgren/Lipscomb method, the minimization path stops at an intermediate point 150 and a curved transition path 160 is defined using the intermediate point 150, the reactant 120 and product 130 to determine a QST path 160. The maximum energy point 155 on this QST path 160 is a refined estimate for the transition state geometry and energy.

The LST and QST approach provide a method to approximately locate the positions of transition states. However, robust and efficient methods of refining transition states to improved levels of precision that are applicable to more complex systems are needed.

### SUMMARY OF THE INVENTION

In one embodiment, the invention comprises a method of estimating an intermediate state geometry of interacting atoms at a point between an initial reactant state and a final product state. The method may comprise defining a set of interpolated atomic distances between at least some atoms of the system and comparing at least some of the interpolated atomic distances to a threshold value. The method further comprises defining a set of atomic coordinates by minimizing a sum, wherein at least some terms of the sum correspond to distances between atom pairs of the system and include a measure of the difference between an interpolated atomic distance between an atom pair and an atomic distance between the same atom pair derived from the set of atomic coordinates; wherein only terms that correspond to atom pairs having an interpolated atomic distance smaller than the cutoff value are included in the sum. The method may be advantageously applied when the atomic system comprises a repeating periodic structure. In some embodiments, the repeating periodic structure comprises a set of repeating unit cells, and terms of the sum have different weight depending on which unit cell at least one atom of the atom pair occupies.

In another embodiment, the invention comprises a method of estimating a transition state geometry of a system of interacting atoms, wherein the system comprises a periodic structure of a repeating set of unit cells. The method comprises using a geometric analysis of interpolated atom pair separations so as to define a set of atomic coordinates for atoms of the system in a state intermediate between an initial reactant state and a final product state, wherein the geometric analysis comprises determining a unit cell in which at least one atom of an atom pair resides, and wherein the geometric analysis depends on the determination.

In still another embodiment, a method of estimating a transition state geometry of a periodic system of interacting atoms defining a repeating set of unit cells comprises weighting the effect on transition state geometry of atom position in one of the unit cells of the repeating set more than atom position in other unit cells of the repeating set.

Systems for performing transition state geometry calculations are also provided. In one embodiment, such a system comprises a memory storing atomic coordinates corresponding to the initial reactant configuration and the final product configuration, and means for retrieving the configurations from memory. The system also advantageously includes means for computing interpolated atomic distances between the configurations, means for comparing at least some of the interpolated atomic distances to a threshold value, means for estimating three dimensional atomic coordinates of the set of atoms between the configurations, and means for defining a specific set of atomic coordinates at a selected point between the configurations by minimizing a sum, wherein at least some terms of the sum correspond to distances between atom pairs of the system and include a measure of the difference between an interpolated atomic distance between an atom pair and an atomic distance between the same atom pair derived from estimated three dimensional atomic coordinates; wherein only terms that correspond to atom pairs having an interpolated atomic distance smaller than the cutoff value are included in the sum.

### BRIEF DESCRIPTION OF THE DRAWINGS

FIG. 1 is a graph illustrating the transition of two reactants through an intermediate transition state to a product, including a potential energy graph corresponding to the chemical process.

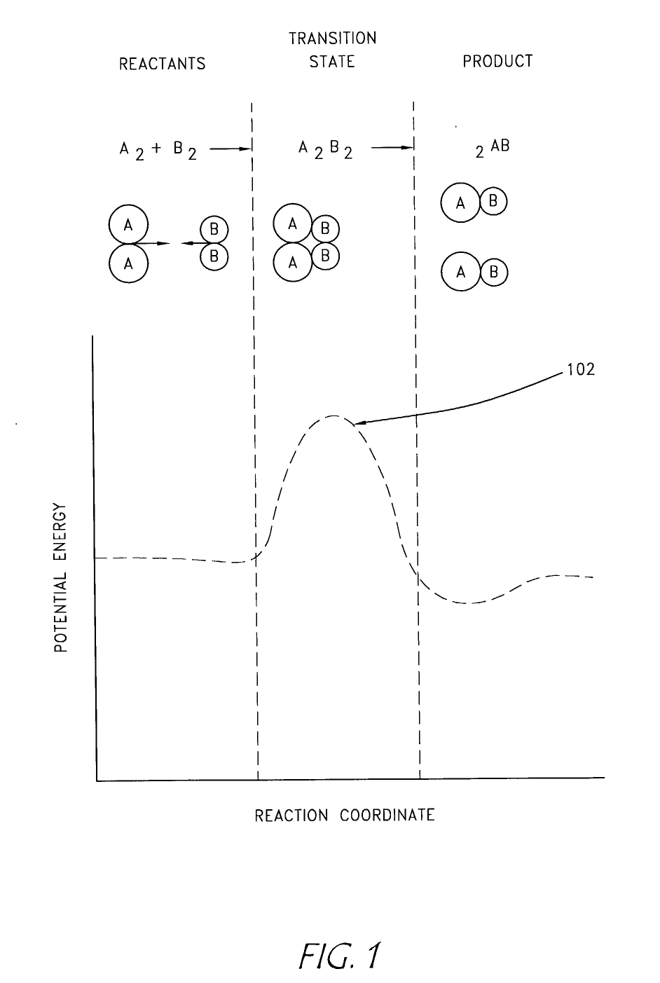

FIG. 2 is a schematic illustrating the LST and QST approach to transition state searching in three dimensions.

FIG. 3 is a schematic illustrating the LST and QST approach to transition state searching in three dimensions utilizing minimizations in the conjugate direction.

FIG. 4 is an example of periodic reactant and product structures.

FIG. 5 is a flowchart illustrating the process of extending the LST and QST approaches to periodic structures.

FIG. 6 is a schematic illustrating the LST and QST approach to transition state searching in two dimensions.

### DETAILED DESCRIPTION OF THE PREFERRED EMBODIMENT

Embodiments of the invention will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments of the invention. Furthermore, embodiments of the invention may include several novel features, no single one of which is solely responsible for its desirable attributes or which is essential to practicing the inventions herein described.

Embodiments of the invention thus include methods of estimating transition state energies and/or transition state configurations. The invention further includes systems comprising a general purpose computer which is configured with one or more software modules to perform one or more of the methods. The general purpose computer will typically include a memory storing instructions and a memory storing atomic configuration information which is retrieved and processed in accordance with the advantageous methods described herein. The invention also includes computer readable media having instructions stored thereon which cause a general purpose computer to perform one or more of the inventive methods.

In an article by Bell and Crighton [S. Bell and J. S. Crighton, J. Chem. Phys. 80, 2464 (1984)], which is hereby incorporated by reference for all purposes, the concept that transition state structures emerging from an LST or QST calculation can be refined using conjugate gradient methods was introduced. The conjugate gradient method allows transition state optimization without calculating the second derivatives of the energy with respect to the atomic coordinates and is therefore much faster than traditional methods such as eigenvector following.

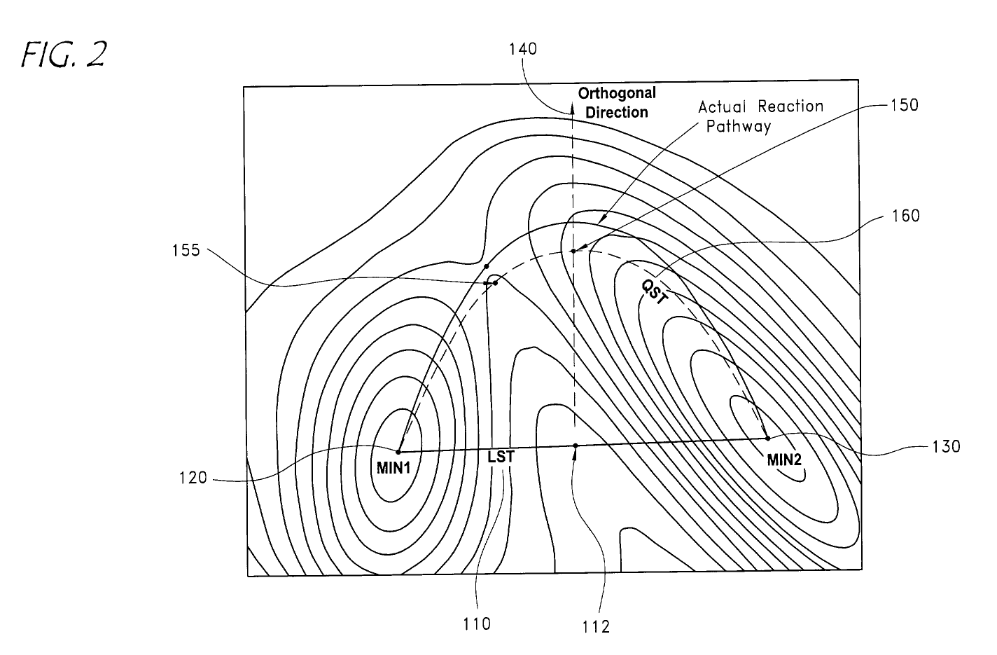

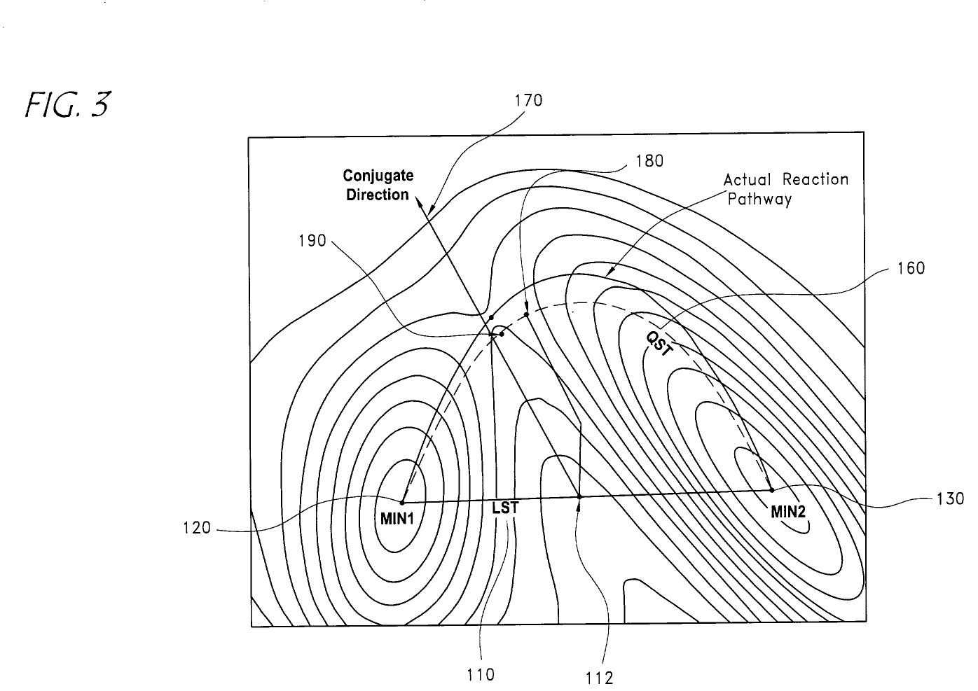

FIG. 3 is a schematic illustrating the LST and QST approach to transition state searching in three dimensions utilizing minimizations in the conjugate direction. The transition state search technique begins with a maximization of the energy along the LST path 110 for the reaction. This LST estimate of the transition state 112 is refined by performing a conjugate gradient minimization 170 (not an orthogonal minimization, as discussed with respect to FIG. 2). As shown in FIG. 3, the conjugate minimization path 170 follows the ridge in the energy surface more closely than when minimizations in the orthogonal direction (e.g. FIG. 2) are performed. A QST calculation is then performed using the reactant 120 and product structures 130 as the end points and the final structure 180 obtained from the conjugate gradient algorithm to define the midpoint. A new conjugate gradient optimization is initiated starting from the QST energy maximum 190. The latter two steps are repeated until convergence is obtained.

The LST method is typically applied to a set of atoms that begin in a “reactant” configuration, and end in a “product” configuration. Inter-atomic distances are calculated at points between the reactant state and the product state by linearly interpolating the distances between all pairs of atoms between reactant and product to determine an intermediate distance

$${r}_{ab}^{i} ( f )$$

according to

$${r}_{ab}^{i} ( f ) = ( 1 - f ) {r}_{ab}^{R} + {fr}_{ab}^{P} , \qquad Equation 1$$

where

$${r}_{ab}^{R} and {r}_{ab}^{P}$$

are the inter-nuclear distances between any two atoms a and b in the reactant(s) and product(s), respectively. The atoms a and b may both be in a single reactant molecule or in separate reactant molecules. In Equation 1, f represents an interpolation parameter that varies between 0 and 1 so that when f=0,

$${r}_{ab}^{i} ( f ) = {r}_{ab}^{R} ,$$

when f=1,

$${r}_{ab}^{i} ( f ) = {r}_{ab}^{P} ,$$

and when 0<f<1,

$${r}_{ab}^{i} ( f )$$

is somewhere between

$${r}_{ab}^{R} and {r}_{ab}^{P} .$$

For example, with respect to particular a and b atoms, if

$${r}_{ab}^{R} = 2 and {r}_{ab}^{P} = 22 then {r}_{ab}^{i} ( .1 ) = 4 , {r}_{ab}^{i} ( .2 ) = 6 …$$

and (0.9)=20. Thus, the intermediate distance

$${r}_{ab}^{i} ( f ) ,$$

corresponding to any two atoms a and b, may be calculated for any atom pair as a function of the value of parameter f.

In a molecule having N atoms, the total number of inter-atomic separations is N×(N−1)/2, which is usually greater than the 3×N Cartesian degrees of freedom of the system. As a result, when N is greater than 7, which is usually the case, Equation 1 over-specifies the geometry of the system. Therefore, in one embodiment, configurations along the LST path are defined by finding atomic coordinates that produce inter-atomic distances which provide the best overall match of the system geometry with the values derived from Equation 1. These may be found by minimizing the function S with respect to r_{ab} as defined in Equation 2:

$$S ( f ) = \frac{1}{2} {\sum }_{a \neq b} \frac{{( {r}_{ab} - {r}_{ab}^{i} ( f ) )}^{2}}{{( {r}_{ab}^{i} ( f ) )}^{4}} \qquad Equation 2$$

In Equation 2, r_{ab} is an inter-atomic distance derived from estimated three dimensional Cartesian coordinates of the particular a and b atoms. In one embodiment, a Cartesian (i.e. X,Y,Z) estimate of the position of the a and b atoms at a particular f is converted to an atomic distance r_{ab} between the particular a and b atoms. The term

$${r}_{ab}^{i}$$

is an interpolated distance along the linear path between the particular a and b atoms at the particular f computed with Equation 1. In an advantageous embodiment, for each particular f, the Cartesian estimate (from which r_{ab} is derived) is iteratively adjusted to minimize the sum of the differences between r_{ab} and

$${r}_{ab}^{i}$$

for all combinations of a and b. The denominator of Equation 2 provides a weighting function which ensures that smaller

$${r}_{ab}^{i}$$

distances are more significant in the sum than larger

$${r}_{ab}^{i}$$

distances. The function S is always greater than or equal to zero, and the reactant and product geometries therefore minimize S when f is 0 and 1, respectively.

Thus, for each selected value for the interpolation parameter f a set of atomic Cartesian coordinates on the LST path may be produced. For example, if f is allowed to vary in steps of 0.1 (i.e. f=0.1, 0.2, 0.3, 0.4, 0.5, 0.6, 0.7, 0.8, or .9) the minimization of S(f) will result in 9 different structures defining an LST path between the reactant and product structures. Note that structures along the LST path are constructed on the basis of geometric analysis alone; no energy calculations are used to obtain them.

In some embodiments the function S(f) may also incorporate terms that make the minimization more stable numerically. Equation 3 is one example.

$$S ( f ) = \frac{1}{2} {\sum }_{a \neq b} \frac{{( {r}_{ab} - {r}_{ab}^{i} ( f ) )}^{2}}{{( {r}_{ab}^{i} ( f ) )}^{4}} + {10}^{- 6} {\sum }_{x , y , z} {\sum }_{a} {( {x}_{a} - {x}_{a}^{i} ( f ) )}^{2} , \qquad Equation 3$$

where xₐⁱ is an interpolated Cartesian coordinate of an atom at a specific interpolation factor f, and Xₐ is the Cartesian coordinate being determined through the minimization. The 10⁻⁶ term weights these terms very small with respect to the remainder of the equation. This additional sum prevents the Cartesian coordinates from rotating or translating arbitrarily.

As discussed above, one approach to estimating transition point energies involves the use of conjugate gradient methods, which are often used to locate saddle points of functions. Conjugate gradient methods make intelligent use of the gradient information that is available on each cycle of the optimization process and are generally much more efficient than steepest descent methods. An important feature of these methods is that the conjugate directions can be constructed using gradient information that is generated in the course of the optimization process; at no stage is it necessary to calculate the Hessian matrix.

In practice, the energy surface for a reaction is only approximately quadratic, and naive application of a conjugate-gradient saddle-point algorithm will usually prove unsuccessful because the system will have a tendency to fall to a point on the energy surface below the saddle. Repeated application of the conjugate gradient minimization will often optimize the system to a local minimum in the vicinity of the transition state rather than the saddle point itself. A tendency of the optimization process to veer off the ridge of the energy surface and toward a minimum will manifest itself as a build up of the gradient in the first direction. In a practical scheme the conjugate gradient process must be restarted with a new maximization step if the gradient in the first direction becomes too large.

If the gradient on all atoms becomes smaller than a given threshold during the conjugate gradient optimization, then the search is successful and the optimization halts. Conversely, if the gradient in the first direction (i.e., the direction defined by the tangent to the LST) becomes large or if a complete set of conjugate directions are searched unsuccessfully, then a QST calculation is performed using the reactant, the product, and the best guess obtained from the conjugate gradient algorithm. This procedure is similar to that used to compute the LST, but uses an equation which is quadratic in the interpolation parameter f rather than linear in f to produce the initial set of interpolated interatomic distances.

When the point of maximum energy along this QST path is located, a new conjugate gradient optimization is initiated using the tangent to the QST trajectory at the energy maximum to define a new direction. Cycles of QST maximization and conjugate gradient refinement may be repeated until convergence is obtained or some maximum desired number of QST cycles is reached.

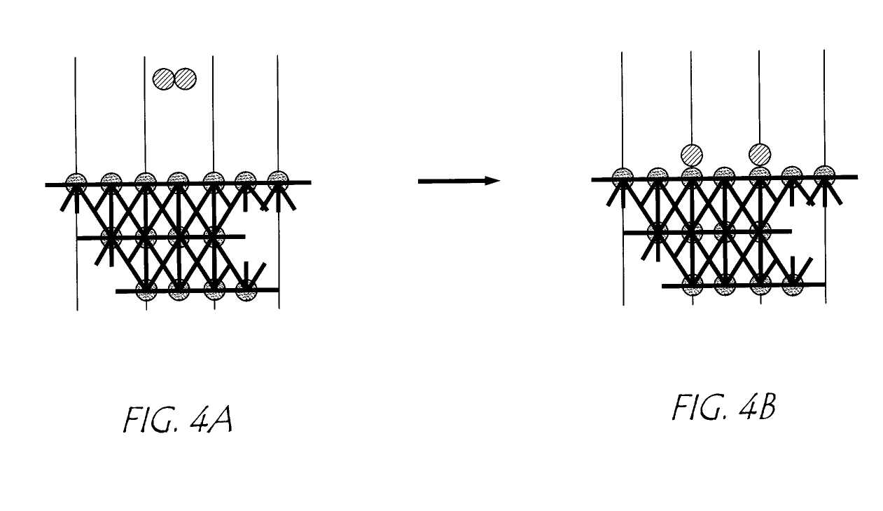

The above described methods of finding transition structures have been applied with some success to reactants comprising finite atomic systems. However, there is a need for a method of finding transition state structures when one or both reactants is a periodic atomic system such as a crystal surface. One example of such a system is illustrated in FIGS. 4A and 4B. FIG. 4A illustrates a reactant configuration for a hydrogen molecule near a {111} crystal face of Cu. FIG. 4B is the product state where the hydrogen atoms of the molecule disassociate from each other and bind the Cu crystal surface.

In periodic systems, LST and QST paths may still be defined by a function which is linearand quadratic, respectively, in f as described above. However, calculation of the atomic coordinates by minimizing the sums of Equation 2 or 3 is difficult or impossible as the computation of S would involve sums over an infinite number of atoms. Therefore, it is convenient to work with a modified functional form for S which eliminates the infinite sum.

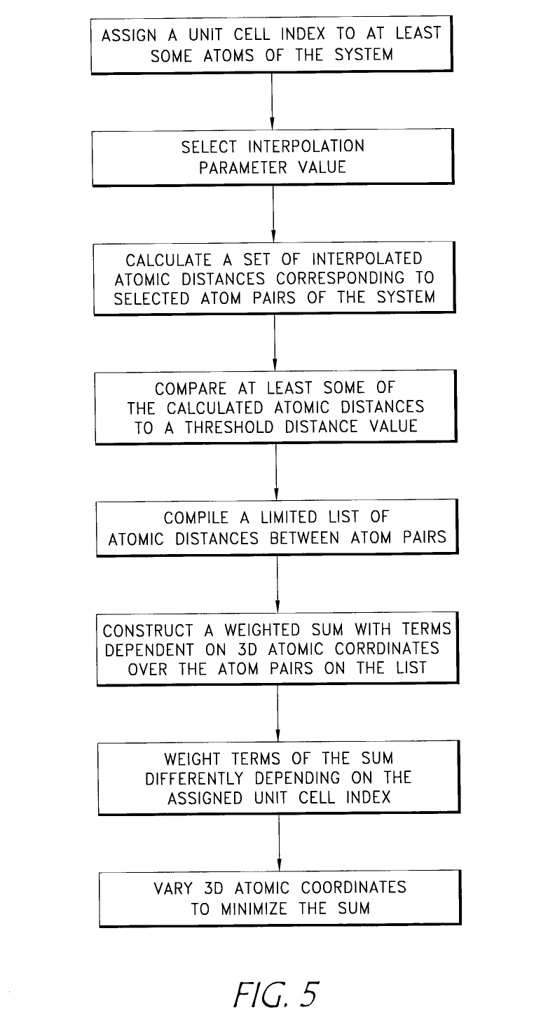

FIG. 5 is a flowchart illustrating the process of extending the LST and QST approaches to periodic structures. This method has been found to be especially useful for modeling reactions that occur on and/or are catalyzed by surfaces. Examples include decomposition reactions such as the decomposition of hydrogen on Cu illustrated in FIG. 4, and the decomposition of methane over Ni. Another specific example which is described in more detail below is the dissociation of NO on a silicon crystal.

When working with a periodic structure, the system being analyzed comprises an asymmetric atomic configuration that regularly repeats in space. The fundamental asymmetric set of atoms is known as the unit cell, and copies of the unit cell are arranged via translations, rotations, or other symmetry operations to form a periodic structure extending through space. Due to the periodic repetition of the unit cell in space, once the geometry of the unit cell is defined, the entire periodic structure geometry may be derived. It has been found that this feature of periodic systems can be exploited to produce an efficient method of estimating transition state geometries for periodic reactants.

In advantageous embodiments for surface related reactions, the initial state of the reaction can be modeled as a regular array of molecular entities such as H₂, CH₃, or NO proximate to an ordered crystalline surface. The product state is another regular array of disassociated molecular entities on the crystalline surface. FIGS. 4A and 4B show one of the hydrogen molecules of the array. Although these examples relate to reactions that occur in association with a surface, there are also reactions involving solids or other three dimensional arrays of atoms for which the methods of analysis described herein are applicable.

Turning now to FIG. 5, the method may begin at block 410 with the definition of the unit cell and the assignment of a unit cell index to at least some atoms of the system. For a surface reaction, each unit cell will typically comprise one surface associated molecular entity and a portion of the underlying crystal of a pre-selected depth. In this case, the system as a whole comprises a planar two-dimensional array of unit cells. A selected unit cell is defined as the origin, and an index R is then assigned to at least some atoms indicating which unit cell the indexed atom resides in. The index R runs over all real space lattice vectors of the periodic structure formed by the reactants. R thus defines an index that is indicative of the number of unit cells in each lattice vector direction that a particular atom of the periodic structure is from the corresponding atom in the unit cell defining the origin. For example, in a two dimensional, orthorhombic periodic structure, R comprises X and Y variables that are indicative of the number of unit cells in the respective X and Y directions the particular atom is away from the corresponding atom in the starting unit cell. If an atom has an index of (0,0) it is in the unit cell at the defined origin of the periodic structure.

The method continues at block 420 when a value for the interpolation parameter f is selected, and for each of a set of atom pairs, an interpolated interatomic distance is computed in a manner analogous to that described above with reference to Equation 1. The set of atom pairs for which interpolated distances are computed and later used in the transition state calculation is advantageously limited, as it will be appreciated that for a periodic structure, an infinite number of interatomic distances are potentially computable. To avoid computing distances for atomic pairs throughout the infinite extent of the array, a limited set of atom pairs is selected for further consideration in the geometric analysis at blocks 430, 440, and 450.

First, interpolated distances at each value of the parameter f may initially be calculated for all atom pairs where both atoms of the pair have an R index of 0. Thus, interatomic distances are calculated for all atom pairs within the origin unit cell. Next, interatomic distances for atom pairs involving atoms within the origin unit cell and atoms within the immediate unit cell neighbors of the origin unit cell may be calculated. For a two-dimensional periodic structure, this would be atom pairs involving atoms of R index (0,0) and the atoms of its eight unit cell neighbors having R index of (0,±1), (±11,0), or (±1,±1). In one embodiment, at block 440, the computed atomic distances for atom pairs where one atom is outside the origin unit cell are compared to a threshold distance value, referred to herein as r_{cut}. Any of these atom pairs having a calculated distance greater than r_{cut} are eliminated from further consideration.

This process of computing interatomic distances between atoms in the origin unit cell and the atoms in other unit cells may be continued for more distant unit cells until it becomes clear that all further calculations will produce distances that are greater than r_{cut}, at which point the procedure can be terminated. With this procedure, at block 450, a list of atom pairs is defined that is limited to those atom pairs wherein both atoms are within the origin unit cell, or wherein one atom is within the origin unit cell and the second atom is in a different unit cell, as long as the distance is less than r_{cut}. Atom pairs in which neither atom resides in the origin unit cell are ignored, regardless of their separation distance.

It may be noted that different systems may be modeled with different unit cell sizes and with different r_{cut} thresholds. For surface reactions, the unit cell dimensions in the surface plane will depend on the selected density of reactant that is on the surface. Unit cell thickness may also be varied in these embodiments. It is generally advantageous for rut to be at least as large as the largest unit cell dimension, with 2 to 3 times larger than the largest unit cell dimension having been found suitable in some embodiments. In one embodiment, the cut off radius r_{cut} is set to about 15-25 Bohr, so R runs over a few unit cells. It is contemplated that r_{cut} may be set to any distance, such as 5 Bohr or 50 Bohr, depending on the size of the unit cell, for example. It will be appreciated that r_{cut} should large enough to include all atoms having a significant impact to the course of the chemical reaction.

In an alternative embodiment, the set of atom pairs used for further consideration may be defined only once, and used for all values of the interpolation parameter f. In some of these embodiments, the set of atom pairs consists of (1) all atom pairs within the origin unit cell regardless of whether the distance is above or below rut, and (2) all atom pairs with one atom in the origin unit cell and one atom in a different unit cell if the atom pair has a distance in either the product state or the reactant state (or both) of less than r_{cut} Atom pairs wherein neither atom resides in the origin unit cell are ignored. In this embodiment, the list of atom pairs used in subsequent calculations is only produced once, and is used for all values of parameter f.

At block 460, a weighted sum over the above defined limited list of atom pairs is constructed with terms dependent on the atomic coordinates of the atoms. In one embodiment which is advantageous for systems including a periodic reactant, this sum takes the following form:

$$\begin{matrix}\begin{matrix}S ( f ) = \frac{1}{2} {\sum }_{a , b , R} {( {r}_{ab + R} - {r}_{ab + R}^{i} ( f ) )}^{2} w ( a , b , R , {r}_{ab}^{i} ( f ) ) + \\ {10}^{- 6} {\sum }_{a} {( {x}_{a} - {x}_{a}^{i} ( f ) )}^{2}\end{matrix} & Equation 5 \\ \begin{matrix}w ( {r}_{ab + R}^{i} ( f ) ) = \frac{1}{{( {r}_{ab + R}^{i} ( f ) )}^{4}} \\ when R = 0 for both atoms a and b\end{matrix} & Equation 6 a \\ \begin{matrix}w ( {r}_{ab}^{i} ( f ) ) = \frac{1}{{( {r}_{ab}^{i} ( f ) )}^{4}} - \frac{1}{{r}_{cut}^{4}} \\ when R \neq 0 for one of atom a or atom b\end{matrix} & Equation 6 b\end{matrix}$$

In the above equations, w defines the weight which determines the significance of the respective a and b atom pair to the minimized sum. For instance, the atoms of the unit cell at the defined origin of the periodic structure where R=0 are weighted the highest. Atoms associated with other unit cells are less significant to the definition of atomic coordinates that minimize the sum.

According to Equation 6a, when R=0 for both atoms a and b, the weighting factor is the same as that used in Equations 2 and 3 for a non-periodic system. Thus, when a or b are within the starting unit cell, they are weighted heavily. Equation 6b is used when R≠0 for one of atom a or b such that atom pairs with one atom within the origin unit cell and the other in a different unit cell are weighted less than atom pairs where both reside within the origin unit cell. Thus, and as set forth at block 470 of FIG. 5, the terms of the sum are weighted differently depending on the value of the index R associated with one or both atoms of a given atom pair under consideration.

The above described method of selecting atom pairs for consideration and the weight function w thus ensures that S can be evaluated for periodic systems in a way that includes the most chemically relevant atoms and which reduces and eliminates computations involving atoms and interatomic distances which can be expected to be less useful in the computation of a reaction path over the energy surface. In a manner analogous to conventional methods, by iteratively varying the atomic coordinates at block 480 the value of the sum S is minimized. The coordinates that minimize the sum are used as the atomic coordinates of the system that are associated with the selected parameter f.

The above system has many advantages over other possible methods of limiting the sum S to a finite number of terms. For example, although it would be possible to use the prior art formula of Equation 3, and to define the complete system as one large cluster consisting of a single molecular entity and a large portion of a crystal structure, this is only an approximation to a periodic calculation; creating a cluster large enough to mimic the properties of a true periodic system leads to a very computationally expensive calculation of energy when finding the ridge/saddle point of the energy surface because so many atoms need to be considered. Furthermore, if the prior art formula of Equation 3 is used with a single small unit cell as the complete system, atoms that are important to the reaction pathway are neglected. In contrast, the methods described above allow for full consideration of the significant atomic contributors to the reaction energy and configuration, while at the same time allowing for computationally efficient energy calculations over periodic crystal cells.

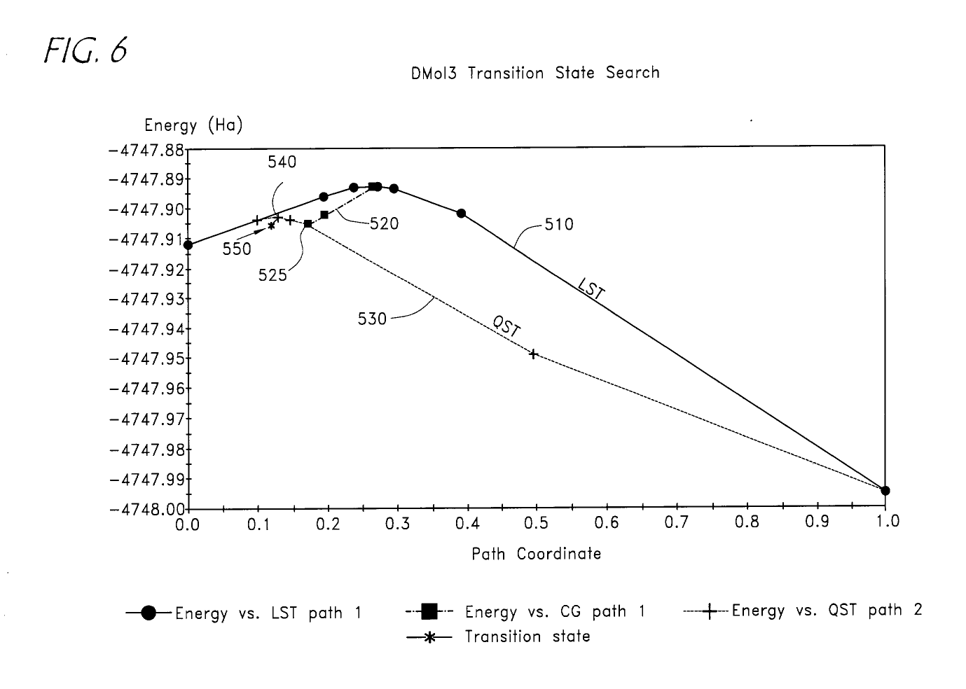

FIG. 6 is a graph illustrating use of the LST and QST approach using the formulas described above to searching for the transition state energy and configuration of the dissociation reaction of NO on the {100} surface of a silicon crystal. In this specific embodiment, the unit cell includes an N atom, an O atom, and 18 silicon atoms, and is 7.68×7.68×19 angstroms. In this embodiment r_{cut} was set to 20 Bohr. The interatomic distances considered in the calculation thus included the N, O, and 18 Si atoms in the origin unit cell, as well as interatomic distances between these origin unit cell atoms and the N, O and Si atoms within the neighboring two to three unit cells of the origin unit cell.

FIG. 6 illustrates the results of the computation, showing the energy along an initial LST pathway 510, followed by conjugate gradient refinement 520 to produce a lower energy point 525 on the energy surface. A QST pathway 530 is calculated using the additional intermediate point 525 on the energy surface. The highest energy point 540 along this QST path is located. Finally, the estimated transition state 550 is located after a second conjugate gradient “walk downhill” from the peak 540 of the QST pathway. While the example of FIG. 5 illustrates two iterations of conjugate gradient refinement, it is contemplated that more or less iterations may be performed to achieve different levels of precision or to utilize more or less computational resources as may be desired in the application.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A method of estimating an intermediate state geometry of interacting atoms at a point between an initial reactant state and a final product state, said method comprising:
defining a set of interpolated atomic distances between at least some atoms of said system;
comparing at least some of said interpolated atomic distances to a threshold value;
defining a set of atomic coordinates by minimizing a sum, wherein at least some terms of said sum correspond to atom pairs of said system and include a measure of the difference between an interpolated atomic distance between an atom pair and an atomic distance between the same atom pair derived from said set of atomic coordinates; wherein only terms that correspond to atom pairs having an interpolated atomic distance smaller than said cutoff value are included in said sum.

2. The method of claim 1, wherein said atomic system comprises a repeating periodic structure.

3. The method of claim 2, wherein said repeating periodic structure comprises a set of repeating unit cells, and wherein terms of said sum have different weight depending on which unit cell at least one atom of said atom pair occupies.

4. The method of claim 1, wherein said interpolated distance comprises a linearly interpolated distance.

5. The method of claim 1, wherein said interpolated distance comprises a quadratically interpolated distance.

6. The method of claim 1, wherein said sum has the form:
$$\begin{matrix}S ( f ) = \frac{1}{2} {\sum }_{a , b , R} {( {r}_{ab + R} - {r}_{ab + R}^{i} ( f ) )}^{2} w ( a , b , R , {r}_{ab}^{i} ( f ) ) + {10}^{- 6} {\sum }_{a} {( {x}_{a} - {x}_{a}^{i} ( f ) )}^{2} , \\ where \\ \begin{matrix}w ( {r}_{ab + R}^{i} ( f ) ) = \frac{1}{{( {r}_{ab + R}^{i} ( f ) )}^{4}} \\ when R = 0 for both atoms a and b\end{matrix} \\ \begin{matrix}w ( {r}_{ab}^{i} ( f ) ) = \frac{1}{{( {r}_{ab}^{i} ( f ) )}^{4}} - \frac{1}{{r}_{cut}^{4}} \\ when R \neq 0 for one of atom a or atom b\end{matrix}\end{matrix}$$
and where R is a multidimensional index indicating a unit cell occupation for a selected atom.

7. A method of estimating a transition state geometry of a system of interacting atoms, wherein said system comprises a periodic structure of a repeating set of unit cells, said method comprising using a geometric analysis of interpolated atom pair separations so as to define a set of atomic coordinates for atoms of said system in a state intermediate between an initial reactant state and a final product state, wherein said geometric analysis comprises determining a unit cell in which at least one atom of an atom pair resides, and wherein said geometric analysis depends on said determination.

8. The method of claim 7, wherein atom pairs of said system which have an interpolated separation greater than a cutoff value are not considered in said geometric analysis.

9. The method of claim 7, wherein said geometric analysis comprises minimizing a sum, wherein at least some terms of said sum correspond to atom pairs of said system and include a measure of the difference between an interpolated atomic distance between an atom pair and an atomic distance between the same atom pair derived from said set of atomic coordinates.

10. A method of estimating a transition state geometry of a system of interacting atoms, wherein said system comprises a periodic structure of a repeating set of unit cells, said method comprising weighting the effect on transition state geometry of atom position in one of said unit cells of said repeating set more than atom position in other unit cells of said repeating set.

11. The method of claim 10, additionally comprising ignoring the contribution to the transition state geometry for interactions involving at least some atoms of said periodic structure.

12. A computer readable medium having stored thereon instructions which cause a general purpose computer to implement a method of estimating a transition state geometry of a chemical system, wherein said method comprises:
defining a set of interpolated atomic distances between at least some atoms of said system;
comparing at least some of said interpolated atomic distances to a threshold value;
defining a set of atomic coordinates by minimizing a sum, wherein at least some terms of said sum correspond to atom pairs of said system and include a measure of the difference between an interpolated atomic distance between an atom pair and an atomic distance between the same atom pair derived from said set of atomic coordinates; wherein only terms that correspond to atom pairs having an interpolated atomic distance smaller than said cutoff value are included in said sum.

13. A method for estimating a transition state geometry of a system of interacting atoms at a point between an initial reactant state and a final product state, said method comprising:
defining a set of interpolated atomic distances between at least some atoms of said system;
assigning an index to at least some atoms of said periodic system, wherein said index is indicative of a position within said periodic structure;
defining a set of atomic coordinates by minimizing a sum, wherein at least some terms of said sum correspond to atom pairs of said system and include a measure of the difference between an interpolated atomic distance between an atom pair and an atomic distance between the same atom pair derived from said set of atomic coordinates; and
weighting at least one term of said sum based on the index associated with at least one atom.

14. The method of claim 13, additionally comprising comparing at least some of said interpolated atomic distances to a threshold value.

15. The method of claim 14, additionally comprising weighting at least one term of said sum based on said comparing.

16. A computer readable medium having stored thereon instructions which cause a general purpose computer to implement a method of estimating a transition state geometry of a chemical system, wherein said method comprises:
defining a set of interpolated atomic distances between at least some atoms of said system;
assigning an index to at least some atoms of said periodic system, wherein said index is indicative of a position within said periodic structure;
defining a set of atomic coordinates by minimizing a sum, wherein at least some terms of said sum correspond to atom pairs of said system and include a measure of the difference between an interpolated atomic distance between an atom pair and an atomic distance between the same atom pair derived from said set of atomic coordinates; and
weighting at least one term of said sum based on an index associated with at least one atom

17. A system for estimating a transition state geometry of a set of atoms between a periodic initial reactant configuration and a periodic final product configuration, said system comprising:
a memory storing atomic coordinates corresponding to said initial reactant configuration and said final product configuration;
means for retrieving said configurations from memory;
means for computing interpolated atomic distances between said configurations;
means for comparing at least some of said interpolated atomic distances to a threshold value;
means for estimating three dimensional atomic coordinates of said set of atoms between said configurations;
means for defining a specific set of atomic coordinates at a selected point between said configurations by minimizing a sum, wherein at least some terms of said sum correspond to atom pairs of said system and include a measure of the difference between an interpolated atomic distance between an atom pair and an atomic distance between the same atom pair derived from estimated three dimensional atomic coordinates; wherein only terms that correspond to atom pairs having an interpolated atomic distance smaller than said cutoff value are included in said sum.

18. The system of claim 17, additionally comprising:
means for defining a unit cell occupation for at least some of said atoms; and
means for weighting terms of said sum differently depending on the defined unit cell occupation.
